(19) United States
(12) Patent Application Publication
(10) Pub. No.: US 2009/0012390 A1
(43) Pub. Date: Jan. 8, 2009

(54) SYSTEM AND METHOD TO IMPROVE ILLUSTRATION OF AN OBJECT WITH RESPECT TO AN IMAGED SUBJECT

(75) Inventors: Jeremie Pescatore, Le Chesnay (FR); Sebastien Gorges, Nancy (FR); Yves L. Trousset, Palaiseau (FR)

Correspondence Address: PETER VOGEL;GE HEALTHCARE, 20225 WATER TOWER BLVD., MAIL STOP W492, BROOKFIELD, WI, 53045, US

(73) Assignee: General Electric Company, Schenectady, NY (US)

(21) Appl. No.: 11/772,350

(22) Filed: Jul. 2, 2007

(51) Int. Cl. A61B 6/03

(52) U.S. Cl. 600/425

(57) ABSTRACT

A system to generate an image dependent on tracking movement of an object travelling through an imaged subject is provided. The system comprises a tracking system operable to detect a position or an orientation of the object travelling through the imaged subject, and an imaging system operable to create a three-dimensional model of a selected anatomical structure of the imaged subject. A controller is operable to store a plurality of computer-readable program instructions for execution by a processor, the plurality of program instructions representative of the steps of: calculating at least one two-dimensional view of a volume of interest extracted from the three-dimensional model, the volume of interest dependent relative to the tracked position of the object, and generating an output image illustrative of the at least one two-dimensional view of the volume of interest.

### BACKGROUND OF THE INVENTION

The subject matter described herein generally relates medical imaging, and in particular to a system and method to guide movement of an instrument or tool through an imaged subject.

Fluoroscopic imaging generally includes acquiring low-dose radiological images of anatomical structures such as the arteries enhanced by injecting a radio-opaque contrast agent into the imaged subject. The acquired fluoroscopic images allow acquisition and illustration of real-time movement of high-contrast materials (e.g., tools, bones, etc.) located in the region of interest 125 of the imaged subject. However, the anatomical structure of the vascular system of the imaged subject is generally not clearly illustrated except for that portion with the injected contrast medium flowing through.

A known technique includes overlaying a three-dimensional image model of a region of interest 125 with a fluoroscopic image of the region of the interest 125, referred to as three-dimensional augmented fluoroscopy, to increase the detail to navigate an object through the imaged subject.

### BRIEF DESCRIPTION OF THE INVENTION

There is a need for an imaging system operable to automatically enhance illustration of an object travelling through imaged subject relative to surrounding anatomical structures of interest and the a tracked location or orientation of the object. There is also a need for an imaging system operable to automatically adapt volume rendering settings of a generated three-dimensional model of imaged anatomical structures of the imaged subject dependent on a location or orientation or both of the object travelling through the imaged subject. There is also a need for an imaging system operable to automatic initialize a position or an orientation of a selected plane of the volume of interest extracted from the three-dimensional model in an interventional context to be displayed for visualization by the operator. The system and method should be applicable not only to augmented fluoroscopy, but as well to other types of imaging systems where the position or orientation of the object 105 travelling through the imaged subject is tracked.

The above-mentioned needs are addressed by the embodiments described herein in the following description.

According to one embodiment, a system to generate an image dependent on tracking movement of an object travelling through an imaged subject is provided. The system comprises a tracking system operable to detect at least one of a position and an orientation of the object travelling through the imaged subject; an imaging system operable to create a three-dimensional model of a selected anatomical structure of the imaged subject; and a controller comprising a memory operable to store a plurality of computer-readable program instructions for execution by a processor, the plurality of program instructions representative of the steps of: calculating at least one two-dimensional view of a volume of interest extracted from the three-dimensional model, the volume of interest dependent relative to the tracked position of the object, and generating an output image illustrative of the at least one two-dimensional view of the volume of interest.

According to another embodiment, a method to track movement of an object travelling through an imaged subject is provided. The method comprises the steps of: a) tracking at least one of a position and an orientation of the object travelling through the imaged subject; b) calculating at least one two-dimensional view of a volume of interest extracted from the three-dimensional model, the volume of interest dependent relative to one of the tracked position and the tracked orientation of the object in step (a); and c) generating an output image illustrative of the at least one two-dimensional view of the volume of interest.

An embodiment of a system to track movement of an object through an imaged subject is also provided. The system includes

### BRIEF DESCRIPTION OF THE DRAWINGS

FIG. 1 is a schematic diagram illustrative of an embodiment of a system to track movement of an object through an imaged subject.

FIG. 2 is a schematic illustration of an embodiment of a method of tracking movement of the object through an imaged subject using the system of FIG. 1.

FIG. 3 is illustrative of localization of an embodiment of an axial, coronal, and sagital cross-section views dependent on a tracked position of the object illustrated in FIG. 1.

FIG. 4 illustrates an embodiment of identified plane(s) extracted from a three-dimensional model dependent on an orientation of the object illustrated in FIG. 1.

FIG. 5 illustrates of an embodiment of an endoscopic view of a volume of interest extracted from a three-dimensional model, the endoscopic view dependent on an orientation of the object illustrated in FIG. 1.

### DETAILED DESCRIPTION OF THE INVENTION

In the following detailed description, reference is made to the accompanying drawings that form a part hereof, and in which is shown by way of illustration specific embodiments, which may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken in a limiting sense.

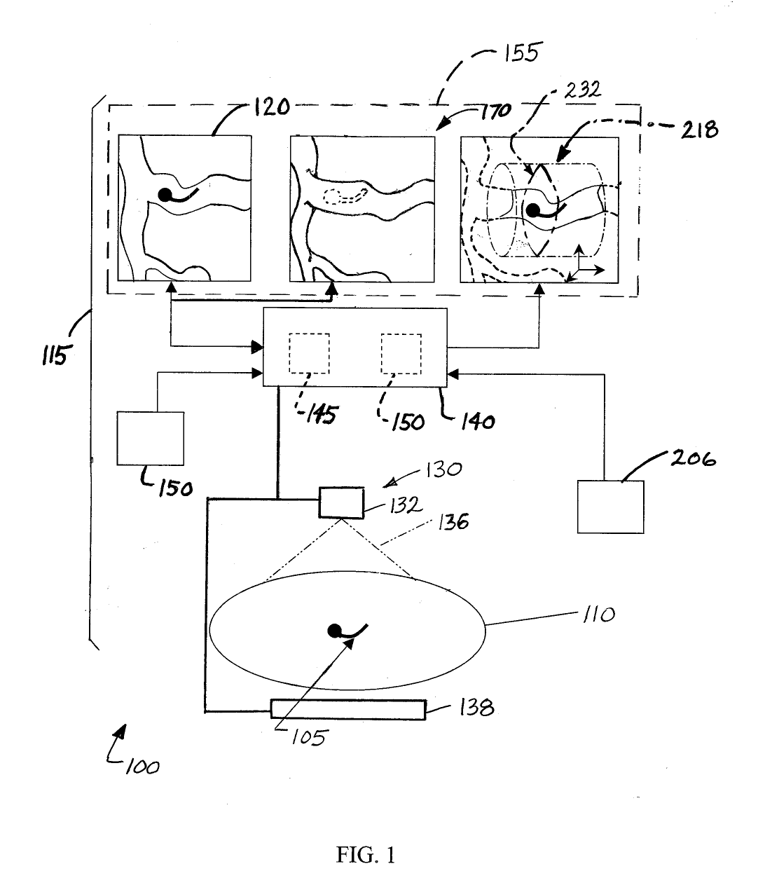

FIG. 1 illustrates an embodiment of a system 100 to track movement or navigation of an image-guided object or tool 105 through an imaged subject 110. The system 100 comprises an imaging system 115 operable to acquire an image or a sequence of images or image frames 120 (e.g., x-ray image, fluoroscopic image, magnetic resonance image, real-time endoscopic image, etc. or combination thereof) illustrative of the location of the object 105 in the imaged subject 110. Thus, it should be understood that reference to the image 120 can include one or a sequence of images or image frames.

One embodiment of the image-guided object or tool 105 includes a catheter or guidewire configured to deploy a stent at a desired position in a vascular vessel structure of the imaged subject 110. Another embodiment of object 105 includes a catheter or guidewire with an ablation device operable in a known manner to selectively destroy tissue or create scar tissue.

The imaging system 115 is generally operable to generate a two-dimensional, three-dimensional, or four-dimensional image data corresponding to a region of interest of the imaged subject 110. The region of interest can vary in shape (e.g., window, polygram, envelope, shape of object 105, etc.) and dimensions. The type of imaging system 115 can include, but is not limited to, computed tomography (CT), magnetic resonance imaging (MRI), x-ray, positron emission tomography (PET), ultrasound, angiographic, fluoroscopic, and the like or combination thereof. The imaging system 115 can be of the type operable to generate static images acquired by static imaging detectors (e.g., CT systems, MRI systems, etc.) prior to a medical procedure, or of the type operable to acquire real-time images with real-time imaging detectors (e.g., angioplastic systems, laparoscopic systems, endoscopic systems, etc.) during the medical procedure. Thus, the types of images can be diagnostic or interventional. One embodiment of the imaging system 115 includes a static image acquiring system in combination with a real-time image acquiring system. Another embodiment of the imaging system 115 is configured to generate a fusion of an image acquired by a CT imaging system with an image acquired by an MR imaging system. This embodiment can be employed in the surgical removal of tumors.

As illustrated in FIG. 1, another embodiment of the imaging system 115 generally includes a fluoroscopic imaging system 130 operable to acquire the images or image frames 120. The fluoroscopic imaging system 130 includes an energy source 132 projecting energy (e.g., x-rays) 136 through the imaged subject 110 to be received at a detector 138 in a conventional manner. The energy is attenuated as it passes through imaged subject 110, until impinging upon the detector 138, generating a fluoroscopic image or frames 120 illustrative of the imaged subject 110. The fluoroscopic imaging system 130 in combination with a software product is generally operable to acquire images or frames 120 for use to generate a three-dimensional, reconstructed image model 170 representative of a region of internal structure or organs of interest of the imaged subject 110. An example of the software product is INNOVA® 3D as manufactured by GENERAL ELECTRIC®. Of course, the software product to generate the three-dimensional model 170 from the series of acquired two-dimensional images 120 can vary.

The image or sequence of acquired image frames 120 and generated models 170 are digitized and communicated to a controller 140 for recording and storage in a memory 145. The controller 140 further includes a processor 150 operable to execute the programmable instructions stored in the memory 145 of the system 100. The programmable instructions are generally configured to instruct the processor 150 to perform image processing on the sequence of acquired images or image frames 120 or models 170 for illustration to the operator. One embodiment of the memory 145 includes a hard-drive of a computer integrated with the system 100. The memory 145 can also include a computer readable storage medium such as a floppy disk, CD, DVD, etc. or other known computer readable medium or combination thereof known in the art.

The controller 140 is also in communication with an input or input device 150 and an output or output device 155. Examples of the input device 150 include a keyboard, joystick, mouse device, touch-screen, pedal assemblies, track ball, light wand, voice control, or similar known input device known in the art. Examples of the output device 155 include an liquid-crystal monitor, a plasma screen, a cathode ray tube monitor, a touch-screen, a printer, audible devices, etc. The input device 150 and output device 155 can be in combination with the imaging system 115, an independent of one another, or combination thereof.

Having generally provided the above-description of the construction of the system 100, the following is a discussion of a method 200 of operating the system 100 to navigate or track movement of the object 105 through the imaged subject 110. It should be understood that the following discussion may discuss acts or steps not required to operate the system 100, and also that operation can include additional steps not described herein. An embodiment of the acts or steps can be in the form of a series of computer-readable program instructions stored in the memory 145 for execution by the processor 150 of the controller 140. A technical effect of the system 100 and method 200 is to enhance visualization of the object 105 relative to other illustrated features of the superimposed, three-dimensional model of the volume of interest 125 of the imaged subject 110. More specifically, a technical effect of the system 100 and method 200 is to enhance illustration of the object 105 without sacrificing contrast in illustration of the three-dimensional reconstructed image or model 170 of the anatomical structure in the volume of interest 125 of the imaged subject 110.

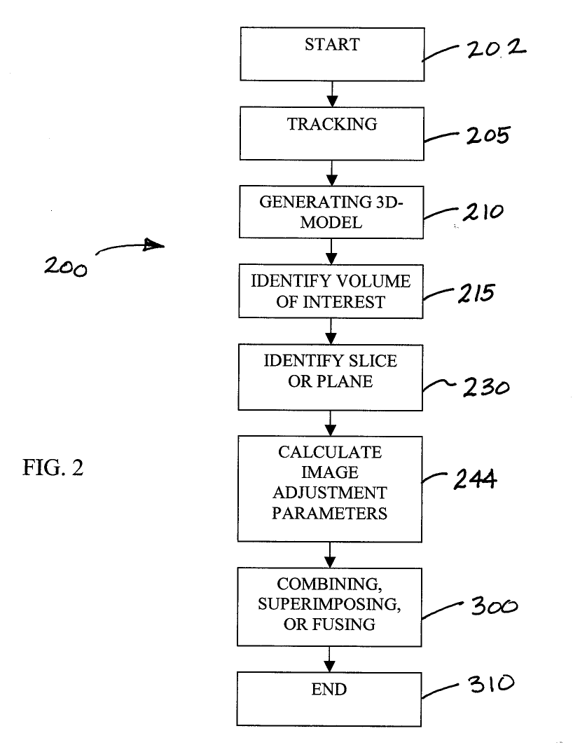

Referring now to FIG. 2, step 202 is the start. Step 205 tracking a location, position or orientation of the object 105 travelling through the imaged subject 110 with a tracking system. One embodiment of the tracking step 205 is performed via known image processing techniques operable to identify voxels or pixels or other captured image data indicative of the object 105 in the one or more of the acquired fluoroscopic images 120 and to calculate its location, orientation or position relative to a coordinate system of the imaging system. This embodiment of the tracking step 205 includes acquiring the two-dimensional, low-radiation dose, fluoroscopic image 120 with the imaging system 115 in a conventional manner of the imaged subject 110. An injected contrast agent can be used to enhance the image 215, but is not necessary with the system 100 and method 200 disclosed herein. Another embodiment of the tracking step 205 can include applying a dilation technique to the fluoroscopic image 120 so as to increase a dimension or size of the imaged object 105 illustrated therein. For example, the object 105 can include a very thin wire that is difficult or too small to identify following superimposition of the fluoroscopic image with the three-dimensional model. To increase the contrast of the object 105, candidate pixels suspected to include image data of the object 105 can be dilated using known techniques of mathematical morphology so as to increase a size of the illustration of the imaged object 105 as captured in the fluoroscopic image 120.

Another embodiment of the tracking step 205 can include calculating or identifying the location or position or orientation of the object 105 via a navigation system 206 (e.g., electromagnetic tracking, optical, etc.) registered in spatial relation relative to the model 170 generated by the fluoroscopic imaging system 130. The tracking step 205 can be updated periodically or continuously with periodic or continuous updates of the fluoroscopic image 120 in real-time, or via the electromagnetic coupling or optical tracking via the navigation system, to measure movement of the object 105 through the imaged subject 110. According to yet another embodiment, tracking movement of the object 105 via image processing techniques applied of the fluoroscopic image 120 can be combined or adjusted to correlate with tracking movement of the object 105 via the navigation system.

Step 210 includes generating or creating the three-dimensional image model 170 from the series of acquired fluoroscopic images 120 with the fluoroscopic imaging system 130.

Step 215 includes automatically identifying or calculating image data of a volume of interest 218 to be extracted from the three-dimensional model 170 correlated to or dependent on the tracked location of the object 105, as described in step 205. The volume of interest 218 generally includes a defined space dependent on or relative to the tracked location of the object 105. Examples of the defined spatial relations include a predetermined radial distance (e.g., a sphere) or other predetermined shape (e.g., cylinder, cube, rectangular box, pyramid, etc.). The defined space can be centered at, or fixed at, or placed at a center or central area in reference to the tracked location of the object 105 as measured or calculated by the tracking system. Image data outside of the volume of interest 218 can be discarded or at least temporarily made transparent. The size of the volume of interest 218 can be predetermined or modified via instructions submitted by the operator through the input device. The volume of interest 218 can be automatically adjusted relative to tracked movement or location of the object 105 relative to the generated model 170. According to another embodiment, the center of the generated volume of interest 218 from the model 170 can be offset by a predetermined spatial relation relative to the tracked location of the object 105.

Referring to FIG. 4, yet another embodiment of step 215 includes identifying, calculating or extracting image data of a volume of interest 218 (e.g., vascular vessel structure or other volumetric portion) of the three-dimensional model 170 identified to include a shared property or within a range of value of a selected parameter. For example, the shared parameter or property of the volume of interest 218 can include the coronary arterial vessel, a carotid artery or vertebral artery structure within a predetermined distance or spatial relation or extending from a starting point relative to the tracked location of the object 105, excluding all other anatomical structures of another property (e.g., bone, etc.) within the defined spatial relation relative to the object 105. In yet another example, the portion of the generated three-dimensional model 170 can include all or a portion of vascular vessel structure that extends from or feeds a volume of interest 218 (e.g., a tumor fed by a nidus of vessels).

Step 230 includes calculating or identifying or extracting image data of one or more plane(s) or slices or cross-sections (e.g., through a vessel) 232 (See FIG. 1) from the volume. An embodiment of the identifying step 230 is correlated or dependent upon a detected position or orientation of the tool or object 105 as described in step 205. The identified position or orientation of the tool or object 105 can be calculated from image processing of the pixel or voxel data illustrative of the object 105 in the fluoroscopic image 138, or according to the navigation system 206, or a combination of both.

Generally, an embodiment of the identifying step 230 includes identifying or calculating a volume rendered two-dimensional display of a projection of the volume of interest 218 extracted from the model 170. The direction of projection can be in a same direction or is relative to a tracked direction or position or orientation of the object 105. This embodiment of step 230 includes computing a volume rendered two-dimensional display of the extracted volume of interest 218 relative to a reference point. The reference point is such that the plane of the monitor or screen or output device illustrating the volume rendered two-dimensional display is generally parallel or orthogonal relative to the identified anatomical structure (e.g., the vessel) containing or including the object 105. In accordance to another embodiment, step 230 generally includes generating the volume rendered two-dimensional view of the three-dimensional model 170 of the volume of interest 218 that projects in a direction from a reference point relative to the detected orientation of the object 105, and is calculated to be one of parallel and orthogonal relative to the orientation of the object 105 in the model 170 of the volume of interest 218.

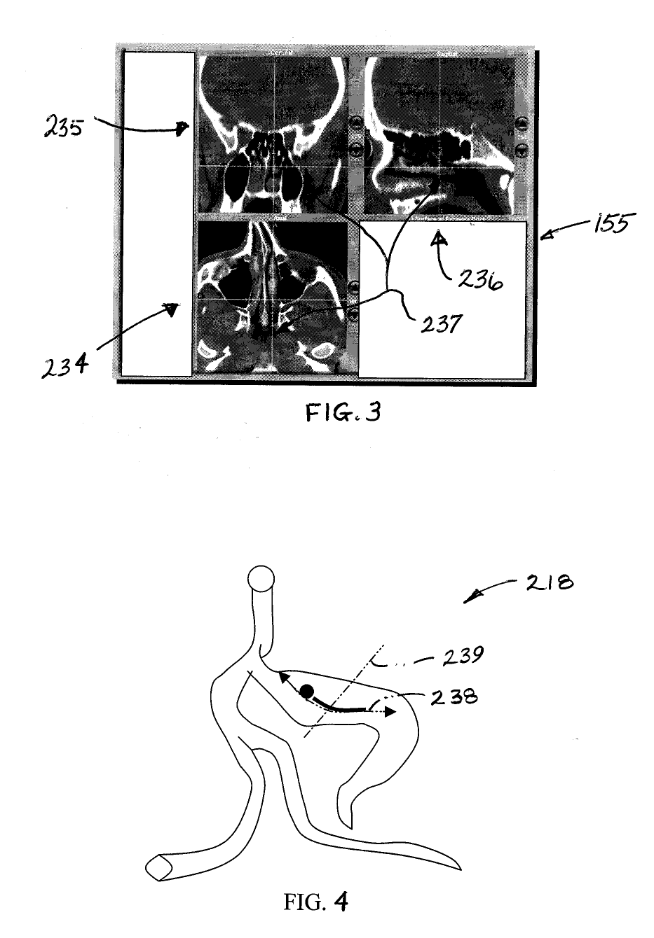

Referring to FIG. 3, a specific embodiment of the identifying step 230 includes calculating or identifying an axial cross-section view 234, a coronal cross-section view 235, and a sagital cross-section view 236 of the volume of interest 218 extracted from the three-dimensional model 170, for illustration to an operator, dependent on or correlated to the tracked location of the object 105 (illustrated by the cursor and reference 237).

Referring to FIG. 4, another embodiment of the identifying step 230 includes calculating or identifying image data along an oblique cross-section or plane 238 extending through the extracted volume of interest 218 dependent on or correlated to the tracked position or orientation of the object 105. For example, the oblique cross-section 238 can be calculated to be in parallel alignment with the tracked orientation of the object 105. In addition or alternatively, an oblique cross-section 239 can be calculated to be orthogonal to the tracked orientation of the object 105.

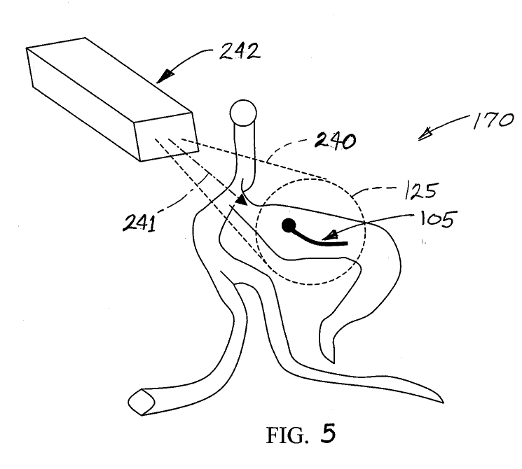

Referring to FIG. 5, another embodiment of the identifying step 230 includes calculating or identifying a two-dimensional, endoscopic view 240 of the model 170 in a direction 241 relative to and extending from an endoscopic starting or vantage point 242 relative to the tracked position or orientation (e.g., alignment having a direction from a first and to a second) of the object 105 as described in step 205.

Step 244 includes calculating image adjustment parameters. Examples of image adjustment parameters include volume rendering parameters associated with generating the plane(s) 232 so as to enhance illustration of the object 105 without reducing detailed illustration of the anatomical structures in the three-dimensional model 170.

There are several rendering parameters that may be identified or altered with respect to generating the plane(s) 232. The projection parameters can depend on the desired information to be highlighted according to image analysis or input from the user.

An example of a projection parameter is a level of transparency of the pixels or voxels comprising the plane(s) 232 of the volume of interest 218 extracted from the three-dimensional model 170 relative to the other. According to one embodiment, the plane(s) 232 are only shown in the output device. According to another embodiment, the planes(s) 232 can be combined, fused, or superimposed with one or more of the acquired fluoroscopic images 120 of the object 105, the volume of interest 218, and the model 170 to create an output image 275 at the output device 155. An embodiment of adjusting the transparency of a pixel by pixel basis includes increasing a value of opacity or contrast or light intensity of each pixel or voxel. For example, a rendering parameter selected or set to about zero percent transparency, referred to as a surface rendering, results in illustration of a surface of the anatomical structure rather than then internalized structures located therein. In comparison, a rendering parameter selected or set to an increased transparency (e.g., seventy percent transparency) results in illustration of detailed imaged data of the internalized structure located therein.

An embodiment of calculating or adjusting a blending parameter according to step 244 includes calculating a value of a blending parameter on a per pixel basis to the slice or plane(s) 232 of the volume of interest 218 extracted from the three-dimensional model 170. The blending parameter or factor generally specifies what proportion of each component (e.g., voxels or pixel data comprising the plane(s) 232 of the volume of interest 218 extracted from the three-dimensional model 170). An embodiment of a blending technique includes applying, identifying, or selecting a blending factor or coefficient that proportions (e.g., linearly, exponentially, etc.) image data (e.g., voxel data, pixel data, opaqueness, shininess, etc.) of the calculated plane(s) 232. An embodiment of a linear blending technique is according to the following mathematical representation or formula:

Fused_image=(alpha factor)*(plane(s) 232 of the volume of interest 218)+(1−alpha factor)*(remainder of the volume of interest 218 extracted from the three-dimensional reconstructed model 170),

where the alpha factor is a first blending coefficient to be multiplied with the measured greyscale, contrast intensity value, etc. for each pixel in the identified plane(s) of the volume of the volume of interest 218, and the (1−alpha factor) is a second blending coefficient to be multiplied with the measured greyscale, contrast, contrast, intensity value, etc. for each pixel of the remainder of the volume of interest 218 not including the identified plane(s) 232.

According to one embodiment of step 244, each of the blending factors is calculated per pixel having a particular x, y, or z coordinate. One or more of the above-described blending factors is applied on a per pixel basis to adjust illustration of the volume rendered plane(s) 232 or remainder of the model 170 as a function according to a two- or three-dimensional coordinate system identified in reference to the three-dimensional model 170. This embodiment of step 244 can be represented by the following mathematical representation:

alpha factor=f(x,y),

where the alpha factor is a blending factor associated each pixel where (x) and (y) represent coordinates in a coordinate system defining a common reference of a spatial relation of each pixel of the the plane(s) 232 of volume of interest extracted from the three-dimensional model 170.

According to an example of this embodiment, step 244 includes identifying and applying a first blending factor alpha to calculate the greyscale, contrast, or intensity values of the pixels of comprising the plane(s) 232 in the three-dimensional model 170 of the volume of interest 218 projecting in combination, fusion or superposition within the fluoroscopic image 138 to create the output image 275. Step 244 further includes identifying and applying or multiplying a second blending factor (the second blending factor lower relative to the first blending factor) to calculate the greyscale, contrast, or intensity values per pixel of the remaining pixels or voxels in the three-dimensional model 170 not included in the plane(s) 232. The step 244 can be performed periodically or continuously in real-time as the object 105 moves through the imaged subject 110 as tracked from image processing of the fluoroscopic image 138 or via the navigation system 206.

It should be understood that other known image processing techniques to vary volume rendering of the plane(s) 232 of the three-dimensional model 170 can be used in combination with the system 100 and method 200 described above. Accordingly, the step 244 can include identifying and applying a combination of the above-described techniques in varying or adjusting values of various volume rendering or projection parameters (e.g., transparency, intensity, opacity, blending) on a pixel by pixel basis or a coordinate basis (e.g., x-y coordinate system, polar coordinate system, etc.) of the calculated plane(s) 232 of the volume of interest 218 of the three-dimensional model 170.

Although not required, step 300 includes combining, superimposing, or fusing the image data of the calculated plane(s) 232 of the volume of interest 218 extracted from the three-dimensional model 170 adjusted as described above in step 230 with the image data of the two-dimensional fluoroscopic image 138 adjusted to better enhance contrast or the object 105 so to create the output image 275 illustrative of the object 105 in spatial relation to the identified plane(s) 232 of the volume of the interest 218. An embodiment of step 300 includes combining, fusing or superimposing one of the fluoroscopic images 120 with a two-dimensional, volume rendered illustration of the calculated plane(s) 232 of the volume of interest extracted from the model 170. Step 310 is the end.

A technical effect of the above-described method 200 and system 100 is to automatically enhance illustration of the volume of interest 218 extracted from the three-dimensional model 170 of the anatomy of the imaged subject 110 relative to a tracked location or orientation of the object 105 moving through the imaged subject 110. Another technical effect of the described method 200 and system 100 is to automatically adapt the three-dimensional volume rendering settings of the generated three-dimensional model 170 dependent on a location or orientation of the object 105. The system 100 and method 200 also provide automatic initialization of the position or orientation of selected plane(s) 232 of the volume of interest 218 extracted from the three-dimensional model 170 in an interventional context. Although the system 100 and method 200 are described with respect to augmented fluoroscopy, it should be understood to those skilled in the art that the system 100 and method 200 are applicable to other types of imaging systems 115 where the position or orientation of the object 105 travelling through the imaged subject 110 is tracked.

This written description uses examples to disclose the invention, including the best mode, and also to enable any person skilled in the art to make and use the invention. The scope of the subject matter described herein is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

We claim:

1. A system to generate an image dependent on tracking movement of an object travelling through an imaged subject, comprising:
a tracking system operable to detect at least one of a position and an orientation of the object travelling through the imaged subject;
an imaging system operable to create a three-dimensional model of a selected anatomical structure of the imaged subject; and
a controller comprising a memory operable to store a plurality of computer-readable program instructions for execution by a processor, the plurality of program instructions representative of the steps of:
calculating at least one two-dimensional view of a volume of interest extracted from the three-dimensional model, the volume of interest dependent relative to the tracked position of the object, and
generating an output image illustrative of the at least one two-dimensional view of the volume of interest.

2. The system of claim 1, wherein the volume of interest is updated according to one of the group comprising periodically, continuously, and with detection of a movement of the object.

3. The system of claim 1, wherein step of calculating the at least one two-dimensional view includes generating an axial cross-section view, a coronal cross-section view, and a sagital cross-section view of the volume interest relative to the detected position of the object.

4. The system of claim 1, wherein the output image includes varying a value of a volume rendering parameters in generating the display of the at least one two-dimensional view.

5. The system of claim 1, wherein the step of calculating the at least one two-dimensional view includes a step of calculating an oblique cross-section view of the volume of interest correlated to the tracked orientation of the object.

6. The system of claim 5, where the oblique cross-section is calculated to be in parallel alignment relative to the tracked orientation of the object.

7. The system of claim 5, where the oblique cross-section is calculated to be orthogonal relative to the tracked orientation of the object.

8. The system of claim 1, wherein the step of calculating the at least one two-dimensional view includes calculating an endoscopic, two-dimensional view of the model of the volume of interest extending in a direction extending from a starting point of the tracked position of the object.

9. The system of claim 1, wherein the step of calculating the at least one two-dimensional view includes calculating a two-dimensional view of the volume of interest extracted from the three-dimensional model, the two-dimensional view projecting from a direction from a reference point relative to a tracked orientation of the object, the two-dimensional view calculated to be one of parallel and orthogonal relative to the tracked orientation of the object.

10. The system of claim 1, wherein the program instructions further includes the step of:
identifying a first blending coefficient applied to the at least one two-dimensional view of the volume of interest extracted from the three-dimensional model calculated in step (b), and identifying a second blending coefficient different than the first blending coefficient applied to a remainder of the volume of interest, the values of the first and second blending coefficients operable to adjust an illustration of the two-dimensional view to the operator.

11. The system of claim 1, wherein the tracking system is operable to track at least one of the position and the orientation of the object via detection of an image data of the object acquired at the imaging system.

12. The system of claim 1, wherein the tracking system is operable to track at least one of the position and the orientation of the object via an navigation system comprising an electromagnetic field coupling with the object.

13. A method to track movement of an object travelling through an imaged subject, the method comprising the steps of:
a) tracking at least one of a position and an orientation of the object travelling through the imaged subject;
b) calculating at least one two-dimensional view of a volume of interest extracted from the three-dimensional model, the volume of interest dependent relative to one of the tracked position and the tracked orientation of the object in step (a); and
c) generating an output image illustrative of the at least one two-dimensional view of the volume of interest.

14. The method of claim 13, wherein the step of calculating the at least one two-dimensional view includes generating an axial cross-section view, a coronal cross-section view, and a sagital cross-section view through the volume of interest.

15. The method of claim 13, the method further including the step of:
varying a value of one of the group of volume rendering parameters comprising opaqueness and shininess in creating the at least one two-dimensional view.

16. The method of claim 13, wherein the step of calculating the at least one two-dimensional view includes calculating an oblique cross-section through the volume of interest correlated to the detected orientation of the object.

17. The method of claim 16, wherein the oblique cross-section is calculated to be one of in parallel alignment with the detected orientation of the object and orthogonal relative to the detected orientation of the object.

18. The method of claim 13, wherein the step of calculating the at least one two-dimensional view includes calculating an endoscopic, two-dimensional view of the volume of interest extending in a direction extending from a starting point of the detected position of the object.

19. The method of claim 13, wherein the step of calculating the at least one two-dimensional view includes calculating a two-dimensional view of the volume of interest projecting in a direction from a reference point relative to the detected orientation of the object, the two-dimensional view calculated to be one of parallel and orthogonal relative to the detected orientation of the object.

20. The method of claim 13, wherein the step of tracking is performed via at least one of detecting an image data indicative of the object in an acquired image of the imaged subject and detecting variation of an electromagnetic field coupling with the object.
